(19) United States
(12) Patent Application Publication
(10) Pub. No.: US 2005/0177066 A1
(43) Pub. Date: Aug. 11, 2005

(54) Neurological and/or psychological tester

(76) Inventor: Vered Aharonson, Ramat Hasharon (IL)

Correspondence Address: DANIEL J SWIRSKY, PO BOX 2345, BEIT SHEMESH, 99544, IL

(21) Appl. No.: 11/029,656

(22) Filed: Jan. 6, 2005

Related U.S. Application Data
(60) Provisional application No. 60/534,387, filed on Jan. 7, 2004.

(51) Int. Cl. A61B 13/00; A61B 5/00

(52) U.S. Cl. 600/559; 600/558

(57) ABSTRACT

A system for performing neurological and/or psychological tests includes a test editor to generate test scripts, a testing unit to run tests using the test scripts, and an analyzer to analyze the results. The units communicate through a network and store their output in a test database. The testing unit modifies the test according at least to at least one reaction time of a subject. The test editor includes a unit having at least one stimulus-response pair defined therein, an editing unit for a test designer to create a test listing from the at least one stimulus-response pair and a script generator to generate a test script from the test listing. The analyzer analyzes spatial motion, both of the cursor movement and of the fingers as they move across the keyboard.

### CROSS-REFERENCE TO RELATED APPLICATIONS

This application claims benefit from U.S. Provisional Patent Application No. 60/534,387, filed Jan. 7, 2004, which is hereby incorporated by reference.

### FIELD OF THE INVENTION

The present invention relates to neurological and/or psychological testing generally and to computerization of such in particular.

### BACKGROUND OF THE INVENTION

There have been neuropsychological tests for many years. Such tests diagnose neurological and mental disorders and diseases. Specifically, neuropsychological tests are used for the diagnosis of dementia and geriatric mental diseases. Typically, these tests are manually administered and taken. However, the article, “Human-Computer Interaction in the Administration and Analysis of Neuropsychological Tests,” by Vered Aharonson and Amos D. Korczyn, Computer Methods and Programs in Biomedicine (2004), Vol. 73, pp. 43-53, discusses a computerized neuropsychological assessment unit, described in FIG. 1, to which reference is now made.

The unit, labeled 10, includes a computer 12, a tester 14 and an analyzer 16. Tester 10 provides standard neuropsychological diagnosis tasks on a monitor 18 and/or speakers 19 of computer 12. Analyzer 16 measures a subject's presses on a keyboard 20 in response to the tasks. Analyzer 16 determines reaction parameters from the key press data and changes the tasks and instructions in response to the subject's parameters, regulating the complexity as a function of how well the subject responds. Moreover, analyzer 16 analyzes the reaction time data after the subject has finished the tasks to provide performance analysis of the tests.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram illustration of a prior art computerized neuropsychological assessment unit;

FIG. 2 is a block diagram illustration of a neuropsychological testing system, constructed and operative in accordance with the present invention;

FIG. 3 is a block diagram illustration of a test editor 26 forming part of the system of FIG. 2;

FIG. 4 is a block diagram illustration of an exemplary testing unit, forming part of the system of FIG. 2;

FIG. 5 is a flow chart illustration of the operations of an exemplary testing unit, forming part of the system of FIG. 2;

FIGS. 6A, 6B and 6C are schematic illustrations of a cursor movement analysis, useful in understanding the operation of an analyzer forming part of the system of FIG. 2; and

FIG. 7 is a schematic illustration of a simplified keyboard and display, useful in understanding keyboard spatial analysis performed by the analyzer of FIG. 2.

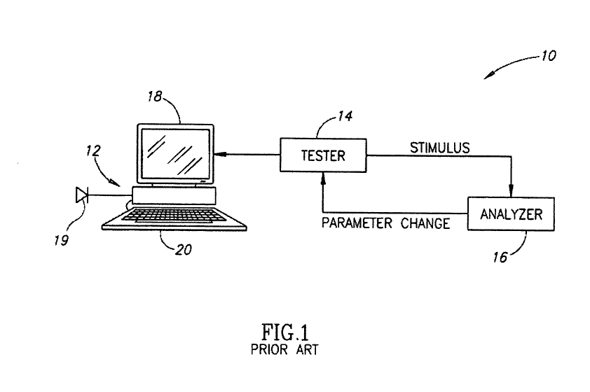

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that the basic paradigm of neurological/psychological tests is very uniform and is as follows: Each test battery consists of a sequence of tests. The sequence and the number of the tests may either be pre-defined by the researcher or dynamically modified during the test flow depending on user's reactions.

Each test may consist of 3 main parts:
1) an explanation;
2) training in the test; and
3) the subtests themselves.

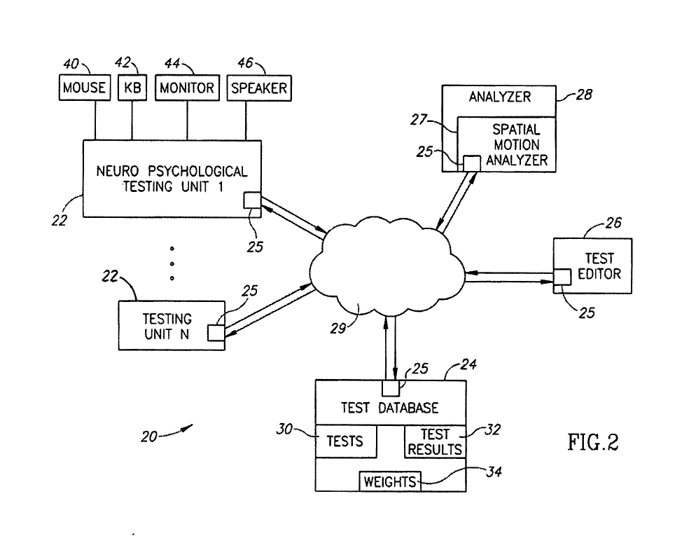

Reference is now made to FIG. 2, which illustrates a neuropsychological testing system 20 which may separate the test design from the execution and/or analysis of the tests. Testing system 20 may comprise a test editor 26 in which to generate the tests, a multiplicity of testing units 22 to run the tests, a test database 24 to store the tests and results, and an analyzer 28 to analyze the results. Because the operations are separate, they may be physically present in separate locations, communicating through a data network 29, such as a local area network, an intranet, or the Internet. In one embodiment, each unit 22, 24, 26 and 28 may comprise a communication unit 25, such as one written in the Java language, through which data may pass from one unit to the next.

Test editor 26 may provide an environment in which to prepare test scripts, such as test scripts 30 stored in test database 24. Each test script may describe a test or series of tests to be performed at one sitting. Each test may comprise a set of explanations, a practice test and the subtests. Each subtest may comprise a set of stimuli (visual or aural), preferably from standardized neuropsychological tests, and a set of questions or actions to be asked of the subject with respect to the stimuli. Included in the subtest definition may be the expected answers and the expected timing of the answers.

The test designer may design explanations in any suitable manner. For example, they may be written explanations to be displayed and/or they may be voiced. The latter may be provided through a recording of someone reading the text or through a text-to-speech device (not shown), such as is commonly known.

The test designer may design the practice test series and may define the passing grade, if necessary, to move to the ‘real’ tests. The test designer may define the type of stimuli and the location in test database 24 where the stimuli may be found. For example, some stimuli may be images. Others might be recordings. The test designer may also associate complexity levels with the stimuli and may have multiple complexity levels for a given test. The complexity levels may be based on the complexity levels in the standardized, manual tests or may be defined by the test designer.

The test designer may also define the expected response to each stimulus. These responses may be key presses, cursor movements and cursor clicks. The expected response may also include the expected timing of the response. For example, the expected response of ‘L’ may be required to be received within 0.25 sec. For cursor movements, the expected response may be defined by an optimal trajectory from the starting location to the final location and by the speed and/or direction at which the cursor may be moved. The test may require cursor clicks to occur within a period of time after the cursor arrives at the location. The test may require that the motion be finished within a predefined length of time.

Attached to each testing unit 22 may be a mouse or other cursor unit 40, a standard or customized keyboard 42, a monitor 44 and a speaker 46. Each testing unit 22 may download a selected test script 30 from test database 24 and may then run test script 30. When running test script 30, testing unit 22 may provide the stimuli listed in test script 30 to a subject and may collect his/her responses. Typical response data may include key presses and cursor movements. They may also include timing of when such occurred with respect to given stimuli.

Testing unit 22 may analyze some of the subject's responses to determine if it is possible to move to more complex stimuli and/or to modify the next expected reaction time. In addition, testing unit 22 may provide the full set of responses as test results 32, typically through data network 29, to database 24.

Analyzer 28 may retrieve tests results 32, through data network 29, and may analyze them at any appropriate time. The analysis may occur at predetermined times after the test has finished, at regular intervals or at any other suitable moment. Analyzer 28 may perform the analysis discussed in the article by Aharonson and Korczyn, discussed hereinabove. Alternatively or in addition, analyzer 28 may perform spatial motion analysis with a spatial motion analyzer 27, operative to analysis the motion of a cursor (such as mouse) and/or the motion of the hands over the keys of a keyboard.

Minimally, analyzer 28 may determine a set of features fᵢ from test results 32 and may determine a score S for each subject. The set of features fᵢ may be those discussed in the article by Aharonson and Korczyn and/or may include cursor movement features fᵢ determined by cursor movement analyzer 27. Score S may be determined by:

$$S = {\sum }_{i} {w}_{i} {f}_{i} < T$$

where T is a threshold defining a disease and wᵢ are empirically determined, per feature weights. The weights wᵢ may be determined for a given population. In one embodiment, the weights were derived from the data of an initial experiment and a follow-up experiment. Through a boost search algorithm, the weights that match best the subjects' cognitive decline towards disease or disorder were calculated. In an alternative embodiment, the weights may be dynamically refined.

The weights may be preferably stored as weights 34 in database 24. Different populations may have different sets of weights 34 and analyzer 28 may select the appropriate set of weights 34 for the subject when performing the analysis.

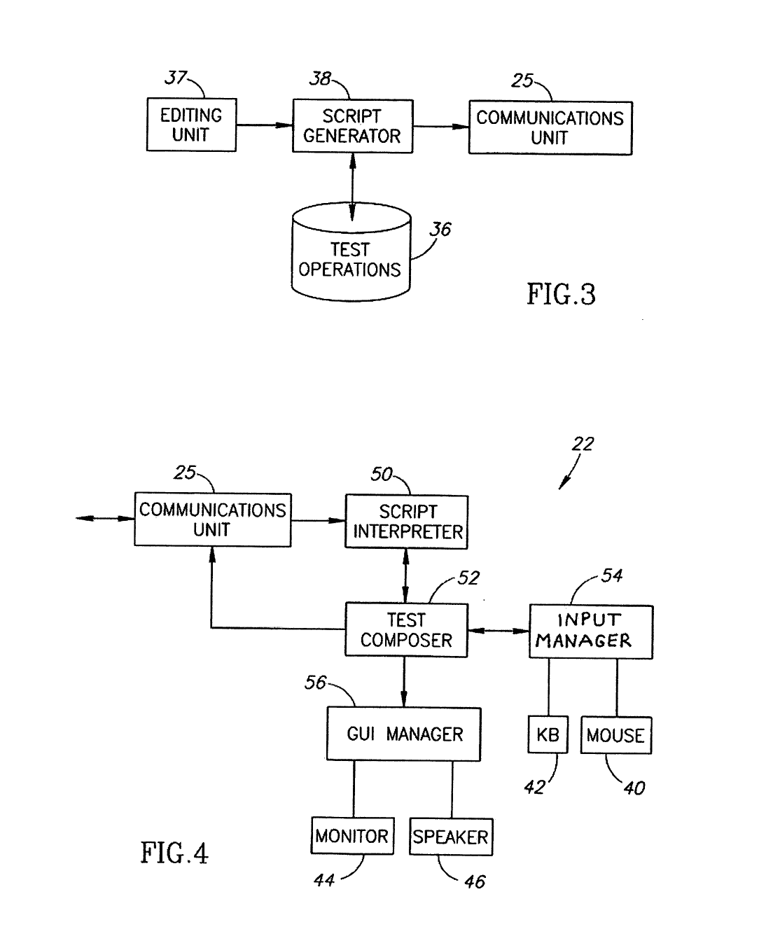

Reference is now made to FIG. 3, which illustrates an exemplary test editor 26. Test editor 26 may comprise a test operation storage unit 36, an editing unit 37, a script generator 38 and one of communication units 25. Within editing unit 37, a test designer may define test batteries, tests, SRPs (stimulus-response pairs, the minimal unit of user/system interaction), test results and subject information.

Each SRP may comprise 2 parts: computer stimuli and their expected user response. Each subtest may be a sequence of SRPs and the stimuli may be sentences of instructions, sentences of explanation, sentences of comments, a visual pattern/symbol/picture, and/or sound or speech.

The test designer may define the amount of stimuli, their types, the desired response for each stimulus and the maximal response time to be allowed. For each stimulus, the test designer may define the stimulus type, the associated audio file, any associated bitmap(s) or a rule (description) for creating the bitmap(s) on the fly, its location on the screen and any rule(s) for selecting the next SRP. For example, the selection rules might be: a random selection, a selection adaptive to user reactions, selections in ascending/descending complexity level, etc. Finally, the test designer may define a format for the results. For example, the test results may be stored as raw data or as summaries.

Test operation storage unit 36 may store code associated with the various types of operations that a test designer may select within editing unit 37.

Script generator 38 may convert the test designer's selections into a test script 30. In the exemplary embodiment, test scripts 30 are XML documents written using an XML Schema. Alternatively, they can be any other suitable document which may be read by testing units 22.

Generator 38 may access storage unit 36 for the code associated with each selection of the test designer. Generator 38 may also add any additional code to generally define the operations to be done.

Reference is now made to FIG. 4, which illustrates an exemplary testing unit 22 which may operate with test scripts written in XML and software written using the Java language. Other forms of operation are possible and are included in the present invention.

Each unit 22 may comprise its communication unit 25, a script interpreter 50, a test composer 52, an input manager 54 and a graphical user interface (GUI) manager 56. Input manager 54 may connect to the input units, such as keyboard 42 and mouse 40. GUI manager 56 may control monitor 44 and speaker 46.

Test composer 52 may run a selected test. To do so, it may first call communication unit 25 to retrieve the specified test script 30 from database 24. Composer 52 may call script interpreter 50 to convert the retrieved test script 30 to a set of Java classes and may then build and run the test with the Java classes. The running of a test is described in more detail hereinbelow, with respect to FIG. 5.

Composer 52 may store the subject's responses during the test battery and may call script interpreter 50 to convert the test results to XML. Finally composer 52 may call communication unit 25 to store the test results in database 24.

Communication unit 25 may be written in Java and may connect each test unit 22 and database 24. It may handle all communication and/or network operations. In addition, it may handle database operations, such as GET and PUT operations, and converting requests from test composer 52 into standard database requests, such as SQL queries. It may also receive query results from database 24 and may pass the results to the request originator.

Script interpreter 50 may convert between test scripts 30, (in this example, written in XML), and a set of programming language classes (in this example, Java). For converting from XML, interpreter 50 may get references to an empty set of Java classes, may run a standard XML parser to convert XML data to Java classes and may return the Java classes to the calling routine For converting to XML, interpreter 50 may get references to a filled set of Java classes, may walk through the classes, extracting data and convert them back to an XML file and may return the XML file to the caller.

Reference is now made to FIG. 5, which illustrates an exemplary operation for running a test battery. For each test in the test battery, there are two training sessions followed by the actual test. The first training session typically may be relatively simple while the second training session may be a more complex version of the same type of task. The actual test may provide multiple tasks of the same type, some simple and others complex.

In step 60, composer 52 may show a welcome screen after which (step 62), composer 52 may get the subject information, typically according to a dialog screen. In step 64, composer 52 may request test script 30 from database 24 (through communication unit 25) and may request that script interpreter 50 convert it. After this set up, composer 52 may run the test.

The test may comprise multiple tasks, which composer 52 may run sequentially in the loop of steps 66-88. For each task, composer 52 may first initiate the task (step 66). In step 68, composer 52 may provide the test explanation, as indicated in test script 30. In step 70, composer 52 may run the first training task, displaying the stimuli defined for it and receiving the subject's responses. If the subject requires another trial (as checked in step 72), composer 52 may review the data and may make (step 74) the task more or less complex to adapt to the subject's responses. Composer 52 may repeat the process (from step 68) until the subject either has mastered the task (according to the definitions in test script 30) or has achieved the maximum number of trials (as listed in test script 30). The check is performed in step 72.

Composer 52 may continue (step 76) with a second training session, using the stimuli defined for it. If the subject requires another trial (as checked in step 78), composer 52 may review the data and may make (step 80) the task more or less complex to adapt to the subject's responses. Composer 52 may repeat the process (from step 76) until the subject either has mastered the task (according to the definitions in test script 30) or has achieved the maximum number of trials (as listed in test script 30). The check is performed in step 78.

Finally, in step 82, composer 52 may provide the test for which the subject has been trained. In this step, composer 52 may take the data and may analyze it to determine when to make the tasks listed therein more complex. Such an analysis is discussed in the above-mentioned article by Aharonson and Korczyn.

After running the test, composer 52 may store the data (step 84), and set up to do the next test, which may either be the next one listed (step 86) or another one later on in test script 30 (step 88). If the test battery has finished, as checked in step 90, composer 52 may analyze and store the results (steps 92 and 94).

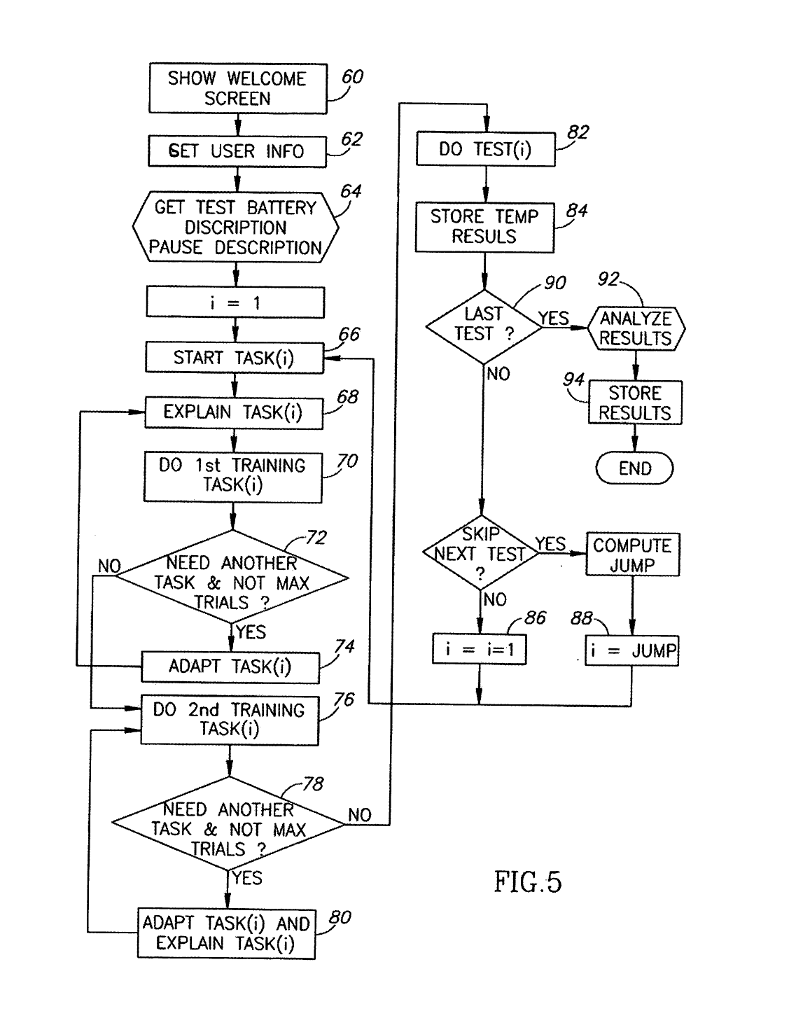

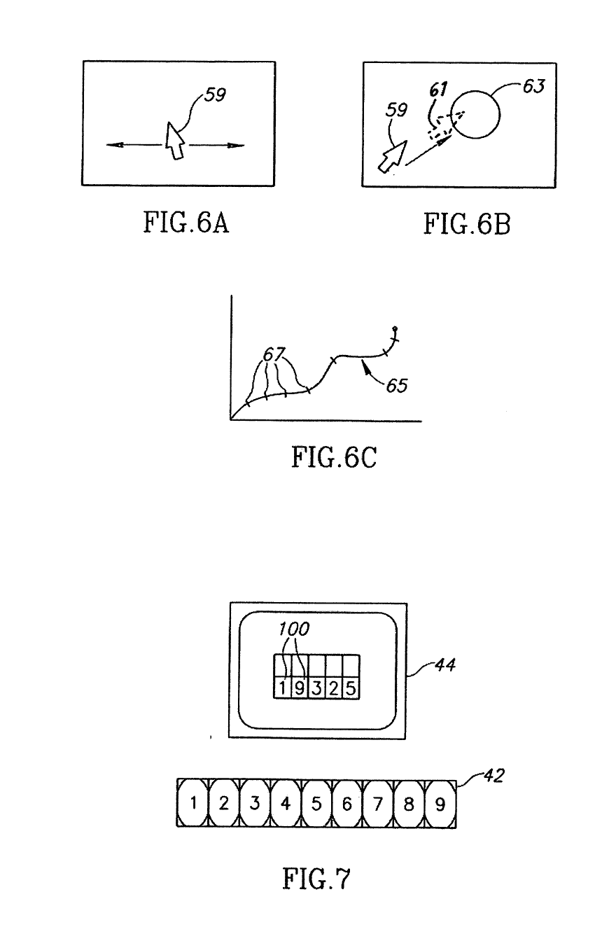

Reference is now made to FIGS. 6A, 6B and 6C, which illustrate aspects of the cursor movement analysis of analyzer 27. FIGS. 6A and 6B illustrate two types of cursor movement tests. In the test of FIG. 6A, the subject may be told to move a cursor 59 back and forth and in the test of FIG. 6B, the subject may be told to move cursor 59 from a starting point 61 to a button 63 and to select button 63, such as by clicking on it. Testing unit 22 may record the cursor trajectories.

Spatial motion analyzer 27 may determine features related to the quality of cursor movement. To do so, analyzer 27 may divide each cursor trajectory, shown in FIG. 6C as a curve 65, into a multiplicity of linear segments 67. For each segment, analyzer 27 may determine the speed, a feature v1, and the variance of the movement from a straight (line. The latter may be a feature v2. Analyzer 27 may then average the values of features v1 and v2 over the line segments 67. Analyzer 27 may determine the jerkiness of the subject's motion as a function of how many segments the trajectory must be divided into.

For the movement of the type of FIG. 6B, spatial motion analyzer 27 may determine the subject's manner of stopping cursor 59 at button 63 (whether in a stable manner or with much stopping) and the location of the stop (a feature v4) with respect to the center of button 63. The stopping manner may be determined by counting the number of crossings in and out of button 63, a feature v3.

Spatial motion analyzer 27 may also determine the click latency (a feature v5) as a measure of how long after the subject brought cursor 59 to button 63 did s/he click button 63. Finally, analyzer 27 may determine click persistency (a feature v6) as a measure of how long the subject pushes on button 63 (i.e. from click on to click off).

Reference is now made to FIG. 7, which is useful in understanding the operation of spatial motion analyzer 27 when analyzing key presses. Testing unit 22 may display an image, on monitor 44, of some numbers 100 for the subject to type using keyboard 42. FIG. 7 shows only the number keys of keyboard 42. As can be seen from FIG. 7, some of the keys, such as the 1 and the 2 keys, are close to each other while other keys, such as the 1 and the 9 key, are further apart.

Applicant has realized that, due to the spatial relationship of the keys, it will take longer to press keys that are apart from each other than those which are nearby. Thus, analyzer 27 may normalize the reaction time data of subsequent key presses as a function of the spatial relationships of the keys to each other. The spatial relationship may be expressed in absolute or relative distance between the keys.

For example, for the keys 100 indicated on monitor 44 of FIG. 7 (i.e. 1, 9, 3, 2 and 5), the reaction times may be normalized so that the relationship of each key to its subsequent key is: 8, 6, 1 and 3, which defines the number of keys on keyboard 42 between subsequent key presses.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A testing unit comprising:
a communication unit to retrieve a test script from a database; and
a test composer to run a neurological and/or psychological test described in said test script, to modify said test according at least to at least one reaction time of a subject and to provide test results to said database through said communication unit.

2. The unit according to claim 1 and also comprising a test script interpreter to convert said test script to code for said test composer to run.

3. The unit according to claim 1 and wherein said test composer comprises means for providing a training period and a testing period to said subject.

4. The unit according to claim 1 and wherein said test composer comprises means for generating a stimulus according to said test script and for comparing a received response to a desired response listed in said test script.

5. A test editor comprising:
a unit having at least one stimulus-response pair defined therein;
an editing unit for a test designer to create a test listing from said at least one stimulus-response pair; and
a script generator to generate a test script from said test listing.

6. The editor according to claim 5 and also comprising a communication unit to provide said test script to a storage unit.

7. The editor according to claim 5 and wherein said test listing comprises at least one stimulus, a desired response for said stimulus, and a maximal allowed response time.

8. The editor according to claim 7 and wherein said test listing comprises, for each stimulus, at least one of the following: a stimulus type, at least one associated audio file, at least one associated bitmap and a rule for selecting a next stimulus-response pair.

9. The editor according to claim 8 and wherein said rule comprises at least one of the following rules: a random selection, a selection adaptive to user reactions and selections in ascending/descending complexity level.

10. An analyzer comprising:
a feature extractor to generate cursor movement features related to errors in cursor motion of a subject performing neurological and/or psychological tests; and
a performance analyzer to determine a cursor motion score from said features.

11. The analyzer according to claim 10 and wherein said features comprise at least one of the following: average speed of a plurality of motion segments; average variance from a straight line of said motion segments, the number of said motion segments, manner of stopping cursor at a displayed button, location of stop with respect to the center of said button, click latency and click persistency.

12. An analyzer comprising:
a reaction time measurer to measure the time a subject performing neurological and/or psychological tests takes to move from a first pressed key to a second pressed key; and
a spatial normalizer to normalize said reaction time as a function of the spatial distance between said keys on a keyboard.

13. A method comprising:
retrieving a test script from a database; and
running a neurological and/or psychological test described in said test script; and
during said running, modifying said test according at least to at least one reaction time of a subject.

14. The method according to claim 13 and also comprising converting said test script to code for said step of running.

15. The method according to claim 13 and wherein said running comprises providing a training period and a testing period to said subject.

16. The method according to claim 13 and wherein said running comprises generating a stimulus according to said test script and comparing a received response to a desired response listed in said test script.

17. A method comprising:
having at least one pre-defined stimulus-response pair;
enabling a test designer to create a test listing from said at least one stimulus-response pair; and
generating a test script from said test listing.

18. The method according to claim 17 and wherein said test listing comprises at least one stimulus, a desired response for said stimulus, and a maximal allowed response time.

19. The method according to claim 18 and wherein said test listing comprises, for each stimulus, at least one of the following: a stimulus type, at least one associated audio file, at least one associated bitmap and a rule for selecting a next stimulus-response pair.

20. The method according to claim 19 and wherein said rule comprises at least one of the following rules: a random selection, a selection adaptive to user reactions and selections in ascending/descending complexity level.

21. A method comprising:
extracting cursor movement features related to errors in cursor motion of a subject performing neurological and/or psychological tests; and
determining a cursor motion score from said features.

22. The method according to claim 21 and wherein said features comprise at least one of the following: average speed of a plurality of motion segments; average variance from a straight line of said motion segments, the number of said motion segments, manner of stopping cursor at a displayed button, location of stop with respect to the center of said button, click latency and click persistency.

23. A method comprising:
measuring the time a subject performing neurological and/or psychological tests takes to move from a first pressed key to a second pressed key; and
normalizing said reaction time as a function of the spatial distance between said keys on a keyboard.
